(19) United States
(12) Patent Application Publication
(10) Pub. No.: US 2026/0020778 A1
(43) Pub. Date: Jan. 22, 2026

(54) TRACKING PERFORMANCE OF MEDICAL PROCEDURES

(71) Applicant: OMNIMED I LLC, Moorestown, NJ (US)

(72) Inventors: Jill Goodwin, Santa Ana, CA (US); Nick Moran, Huntington Beach, CA (US); Robert Brown, Aliso Viejo, CA (US)

(21) Appl. No.: 18/994,792

(22) PCT Filed: Jul. 14, 2023
(86) PCT No.: PCT/US2023/027845

§ 371 Date: Jan. 15, 2025

Related U.S. Application Data
(60) Provisional application No. 63/389,792, filed on Jul. 15, 2022.

(51) Int. Cl. A61B 5/11; A61B 5/00; G06F 16/483; G06N 20/00; G06V 10/82; G06V 20/52

(52) U.S. Cl. CPC A61B 5/1128; A61B 5/6802; G06F 16/483; G06N 20/00; G06V 10/82; G06V 20/52; G06V 2201/034

(57) ABSTRACT

Methods, systems, and apparatus, including medium-encoded computer program products, for tracking performance of medical procedures. The process includes identifying a sterile zone within video feeds of a medical procedure room, where the sterile zone includes a region of pixels that define a three-dimensional physical space. Identifying humans in the room and classifying each identified human as being permitted or not permitted to enter the sterile zone. Generating a skeletal poise model and using the skeletal poise model in correlation with the sensor data to track movement of the human's limbs with respect to the sterile zone. Determining that at least a portion of a skeletal poise model of at least one human classified as not being permitted to enter the sterile zone crossed a boundary of the sterile zone, and in response, causing an alert to be presented on at least one display device located within the medical procedure room.

### TECHNICAL FIELD

The present disclosure relates to systems and methods for tracking, training, and evaluating actions performed in a medical environment (e.g., a surgery room). In some examples, the present disclosure is directed to systems and processes for tracking medical personnel while they perform actions within the medical environment and/or for evaluating biometric actions of personnel against best practices in real time and correcting their actions using an expert system as they perform the actions.

### BACKGROUND

It is well known that adverse patient outcomes in medical environments have a number of complex causal sources leading to over 250,000 deaths annually in the United States. There exists a cause and effect relationship for adverse patient outcomes between the environmental conditions before and during a surgery and what occurs during and after the surgical procedure performed on a patient. These adverse patient outcomes have not sufficiently been addressed with current methods and tools, and previous attempts at leveraging better processes have failed to help prevent many adverse effects.

Traditional solutions aimed at reducing adverse patient outcomes and related readmissions include discharge planning, self-care, and regional anesthesia whenever possible, but further work and data-driven approaches are needed to optimize these interventions. No current solutions have combined sensor based and data driven systems and processes to solve such problems more holistically.

### SUMMARY

This specification relates to systems that combine multiple sensors and devices, including without limitation, edge computers, three-dimensional stereographic cameras, thermal imaging cameras, door sensors, load sensors, temperature and humidity sensors, ultra-wide band sensors, airflow and air particle sensors, and high frequency audio sensors; all of which can be augmented by software comprising artificial intelligence algorithms (e.g., modified “you only look once” (YOLO) models using transfer learning, 1-dimensional and 2-dimensional convolutional neural networks, human pose estimation and human hands and digits position detection via deep neural networks, natural language processing via deep neural networks, multilayer perceptrons, decision trees, and random forest search) and non-AI models (e.g., combination of machine vision image treatment methods, speaker segmentation through audio trace signatures, Fast Fourier Transform, cascade classifier, mahalanobis distance, connected components labelling algorithm) for tracking, detecting, analysing, and evaluating the actions of medical staff and the environmental conditions around them, the combination of which can result in detecting adverse events known to lead to adverse patient outcomes after they undergo a medical procedure.

The present disclosure describes systems for training medical staff to perform surgeries as well as during actual surgeries, the combination of which provides data to enhance the artificial intelligence algorithms over time. Systematic collection of all potential adverse events during training and during actual surgical medical procedures provides a resource of information that is consistently collected, enabling comparison and analytics that subsequently can be used to conduct epidemiologic assessments. The disclosed systems and techniques can provide real-time evaluation that is significantly more accurate than traditional simulated medical procedures, which can be critical in objectively evaluating the skills of a student in order to evaluate performance against a best practice skillset as well as tracking the environmental surrounding of the surgical procedures in a holistic way.

AI-based event data collection and analysis enables the identification of complex latent errors that can be addressed through better training, device design, or surgical methods improvement. The feedback loop between real surgeries and surgical procedure training is an important gap, which the present disclosure is directed to solve.

In general, innovative aspects of the subject matter described in this specification can be embodied in methods that include the actions of identifying at least one sterile zone within video feeds obtained from at least a first stereoscopic camera and a second stereoscopic camera located within a medical procedure room, where the sterile zone includes a region of pixels that define a three-dimensional physical space surrounding a surgical table within the medical procedure room. The method includes identifying, within the video feeds, humans in the medical procedure room and classifying each identified human as being permitted or not permitted to enter the sterile zone based on sensor data received from a sensor worn by the human. The method includes generating, for each identified human within the video feeds, a skeletal poise model and using the skeletal poise model in correlation with the sensor data to track movement of the human's limbs with respect to the sterile zone. The method includes determining that at least a portion of a skeletal poise model of at least one human classified as not being permitted to enter the sterile zone crossed a boundary of the sterile zone within at least one of the video feed, and in response, causing an alert to be presented on at least one display device located within the medical procedure room. Other implementations of this aspect include corresponding systems, apparatus, and computer programs, configured to perform the actions of the methods, encoded on computer storage devices.

These and other implementations can each optionally include one or more of the following features. In some implementations, the sensor data is data from an ultra-wideband sensor worn by the human. In some implementations, classifying each identified human includes: determining an identity of the human from radio frequency identification data received from a badge scan as the human enters a door of the medical procedure room; determining, based on the identity, whether the human is permitted to enter the sterile zone; and associating a metadata tag that indicates whether the human is permitted to enter the sterile zone to the skeletal poise model of the human. In some implementations, classifying each identified human comprises: determining an identity of the human from radio frequency identification data received from a badge scan as the human enters a door of the medical procedure room; determining, based on the identity, whether the human is permitted to enter the sterile zone, and associating a metadata tag that indicates whether the human is permitted to enter the sterile zone with an identifier for a position tracking sensor worn by the human. In some implementations, the position tracking sensor is an ultra-wideband sensor. Some implementations include detecting, within the video feeds, a plurality of medical instruments; and for each medical instrument, identifying a type of the instrument. In some implementations, identifying the type of the instrument includes using a YOLO machine learning model to analyze a region of pixels within one or more frames of the video feeds to identify the type of the instrument. Some implementations include applying a metadata tag to each detected medical instrument to uniquely identify the respective instrument and the type of the instrument; and monitoring the location of each detected instrument by tracking locations of each instrument within the video feed data using an object tracking algorithm in correlation with load sensor data received from one or more load sensors positioned on tables or carts used to temporarily store medical instruments during a medical procedure. Some implementations include detecting that a particular medical instrument fell to a floor of the medical procedure room based on data within the video feeds and audio data received from one or more microphones positioned within the medical procedure room. Some implementations include detecting a potential infection event responsive to tracking movement of the particular medical instrument within the video feeds and determining that the particular medical instrument crossed a boundary of the sterile zone within at least one of the video feeds; and in response, causing an infection alert to be presented on at least one display device located within the medical procedure room.

Some implementations include generating, based on thermal video feeds from at least a first thermal camera and a second thermal camera located within the medical procedure room, a thermal map of the medical procedure room; identifying the sterile zone within the thermal map; identifying a plurality of expected hot spots within the thermal map, the expected hot spots representing regions of elevated thermal emissions that are expected to be within the sterile zone; detecting a new hot spot within the sterile zone; and causing an infection alert to be presented on at least one display device located within the medical procedure room.

Another general aspect can be embodied in a medical procedure tracking system that includes a first stereoscopic camera and a second stereoscopic camera positioned within a medical procedure room, a tracking sensor receiver positioned within the medical procedure room, the tracking sensor receiver configured to interface with tracking sensors to identify positions of the tracking sensors relative to a location of the tracking sensor within the medical procedure room, one or more processors in electronic communication with the first stereoscopic camera and the second stereoscopic camera and the tracking sensor receiver, and one or more tangible, non-transitory media operably connectable to the one or more processors. The media stores instructions that, when executed by the processors, cause the processors to perform operations that include identifying at least one sterile zone within video feeds obtained from the first stereoscopic camera and the second stereoscopic camera located within a medical procedure room, where the sterile zone includes a region of pixels that define a three-dimensional physical space surrounding a surgical table within the medical procedure room. The operations include identifying, within the video feeds, humans in the medical procedure room and classifying each identified human as being permitted or not permitted to enter the sterile zone based on sensor data received from a sensor worn by the human. The operations include generating, for each identified human within the video feeds, a skeletal poise model and using the skeletal poise model in correlation with the sensor data to track movement of the human's limbs with respect to the sterile zone. The operations include determining that at least a portion of a skeletal poise model of at least one human classified as not being permitted to enter the sterile zone crossed a boundary of the sterile zone within at least one of the video feed, and in response, causing an alert to be presented on at least one display device located within the medical procedure room.

Another general aspect can be embodied in a medical procedure room that includes a plurality of stereoscopic cameras, a plurality of thermal cameras, at least one location tracking sensor receiver, at least one microphone, and at least on electronic display The plurality of stereoscopic cameras are arranged within the medical procedure room such that a surgical table within the medical procedure room is visible within a field of view of each stereoscopic camera from a different perspective. The plurality of thermal cameras are arranged within the medical procedure room such that the surgical table is visible within a field of view of each thermal camera from a different perspective. The at least one location tracking sensor receiver is arranged within the medical prosecute room to receive signals from location tracking sensors. The at least one microphone is arranged within the medical procedure room to detect audio from a region surrounding the surgical table. The at least one electronic display is arranged within the medical procedure room such that images displayed on the display are visible from the surgical table. And, each of the a plurality of stereoscopic cameras, the plurality of thermal camera, the at least one location tracking sensor receiver, the at least one microphone, and the at least one electronic display are in electronic communication with one or more computers. Some implementations include an additional stereoscopic camera positioned within the medical procedure room to capture video of a sink located in the medical procedure room within a field of view of the particular camera, and an additional microphone located proximate to the sink.

Particular embodiments of the subject matter described in this specification can be implemented so as to realize one or more of the following advantages. The techniques described below provide a holistic and data-driven approach to monitoring actions performed within medical facilities (e.g., operating rooms) and detecting events that may give rise to infection or other adverse patient outcomes. Implementations may improve the patient outcomes following invasive medical procedures. For example, implementations may prevent events that could cause post operation infections in patients. Implementations may significantly improve sanitation within medical facilities where invasive procedures are performed. Implementations provide a system that is capable of tracking movements of all participants (e.g., doctors, nurses, and other staff), and medical instruments or other objects within a medical facility during an invasive procedure, and detecting events that could give rise to infection.

The details of one or more embodiments of the subject matter described in this specification are set forth in the accompanying drawings and the description below. Other features, aspects, and advantages of the invention will become apparent from the description, the drawings, and the claims.

### BRIEF DESCRIPTION OF THE DRAWINGS

FIG. 1 is a block diagram representing exemplary features of a medical process monitoring or training system according to some embodiments of the present disclosure.

FIG. 2 is floor diagram illustrating an exemplary arrangement of sensors and system components of a medical process monitoring or training system according to some embodiments of the present disclosure.

FIGS. 3 and 4 depict different perspective views of an exemplary room outfitted with sensors and system components of a medical process monitoring according to some embodiments of the present disclosure.

FIGS. 5-10 depict screenshots of exemplary graphical user interface output from a medical process monitoring or training system according to some embodiments of the present disclosure.

FIG. 11 depicts a flowchart of an example process for detecting events within a medical procedure room.

FIG. 12 depicts a block diagram of a computer system that may be applied to any of the computer-implemented methods and other techniques described herein.

Like reference numbers and designations in the various drawings indicate like elements.

### DETAILED DESCRIPTION

Embodiments of the present disclosure are directed at capturing effects having potential causal links to adverse patient outcomes through the use of a combination of many sensory device signals augmented by artificial intelligence algorithms (e.g., modified YOLO models using transfer learning, 1-dimensional and 2-dimensional convolutional neural networks, human pose estimation and human hands and digits position detection via deep neural networks, natural language processing via deep neural networks, multilayer perceptrons, decision trees, and random forest search) and non-AI models (e.g., combination of machine vision image treatment methods, speaker segmentation through audio trace signatures, Fast Fourier Transform, cascade classifier, mahalanobis distance, connected components labeling algorithm) to detect, analyze and recommend corrective actions as adverse events take place in real-time or near real-time within the medical procedure timeframe and encompassing the surrounding vicinity of the surgical room or surgical training room, rather than narrowly focusing on just the surgical procedure table.

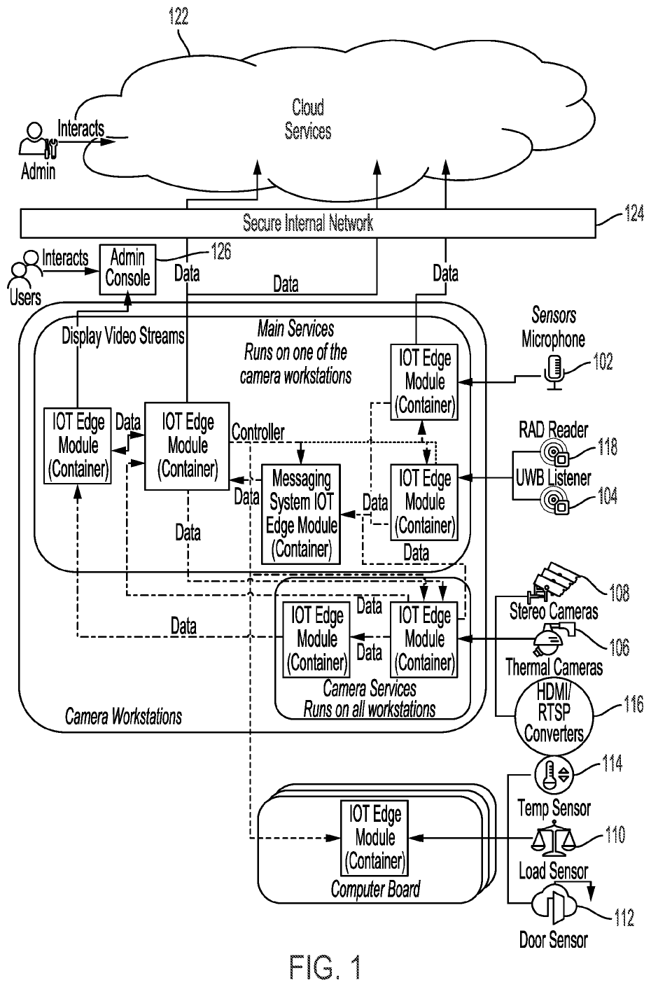

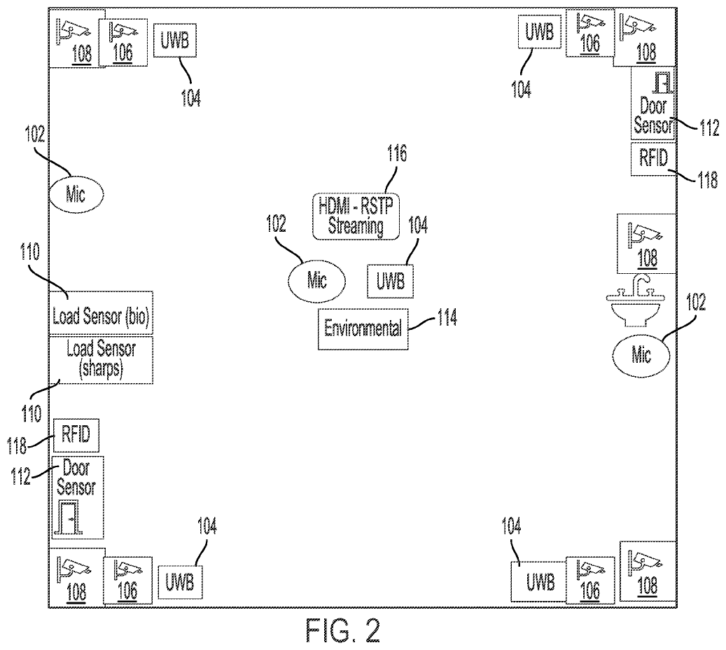

Referring to FIGS. 1 and 2, some embodiments can include a combination of sensors, devices, and other elements, such as microphone(s) 102, real-time locating system (RTLS) tracking sensor receivers and sensors (e.g., ultra-wide band (UWB) anchors and sensors(s)) 104, thermal imaging camera(s) 106, stereographic camera(s) 108, load sensor(s) 110, door sensor(s) 112, environmental sensor(s) 114 that can sense temperature, pressure, humidity, static pressure, dynamic pressure, particles (e.g., between 0.5 μm and 1.0 μm), and carbon-dioxide, high-definition multimedia interface (HDMI) rapid spanning tree protocol (RSTP) wireless streaming device(s) 116 to send and/or receive medical imaging data for display on one or more display devices, and/or radio-frequency identification (RFID) readers 118, all within an AI-based data-driven behavior learning approach along with (optionally) cloud-based and edge-based computing device(s) (e.g., IOT edge modules, messaging systems, sensor APIs, camera APIs, and media servers) that collect and logically assemble cause and effect evidence to ultimately explain adverse patient outcomes and prevent them and/or alarm on adverse contributors in real-time while a surgery is ongoing from an admin console 126, which can be is secured by an internal network 124. Additional sensors are also possible, including for example, air flow sensors and air particle count sensors.

Some embodiments provide a system that can combine its various sensors' data through an aggregator subsystem and apply AI algorithms through a processor to extract and accumulate the following information in real-time during surgical procedures in an operating or training room.

Example operations of the system include detecting specific medical instrument drop events through the combination of feeding and processing of video data streams from cameras 108 to clustering and machine vision algorithms (e.g., combination of continuous video frame differencing and background subtraction, video stream object movement noise filtering with human pose estimation via deep neural networks, video stream object movement noise filtering with density-based spatial clustering of applications with noise (DBSCAN), Gaussian mixture-based background/foreground segmentation for video stream object drop detection in front of other moving objects, Farneback optical flow for video stream movement speed segregation in special cases, image color detection, image segmentation, pixel clustering, pixel thresholding, morphology operations using erode and dilate and/or find contour, video frame processing using cascade filters, windowing, skipping frames, and down sampling, image enhancement using Gaussian blur, kernelling, Laplacian filter, change of color space, image feature detection using Hough Transform, measurement using Euclidean distance and mahalanobis distance, stereovision, histogram, and watershed), and feeding and processing high frequency audio data from microphones 102 to a separate instrument audio signal signature for classification.

Example operations of the system include performing automated medical instrument usage and count through feeding and processing of the video data streams from cameras 108 to machine vision background subtraction, color filtering, and connected components labeling algorithms.

Example operations of the system include classifying medical instruments through feeding and processing of the video data streams from cameras 108 to a trained AI machine vision (e.g., via a modified YOLO model architecture using transfer learning and deep convolutional neural networks) capable of locating and identifying instruments within the video stream.

Example operations of the system include tracking human body motion through the room by feeding and processing of the video data streams from cameras 108 to an AI model via a deep neural network that can associate and estimate a human skeletal pose model and location within the video data stream for gathering accurate human body motion and, in a similar process, also track human hand and finger movement.

Example operations of the system include validating whether the pre-surgery handwashing procedure is satisfactory through feeding and processing of the video data streams from cameras 108 and validating spatial movement and water flow through audio processing (e.g., via 1-dimensional and 2-dimensional convolutional neural networks or Fast Fourier Transform with spectral filtering) from microphone 102.

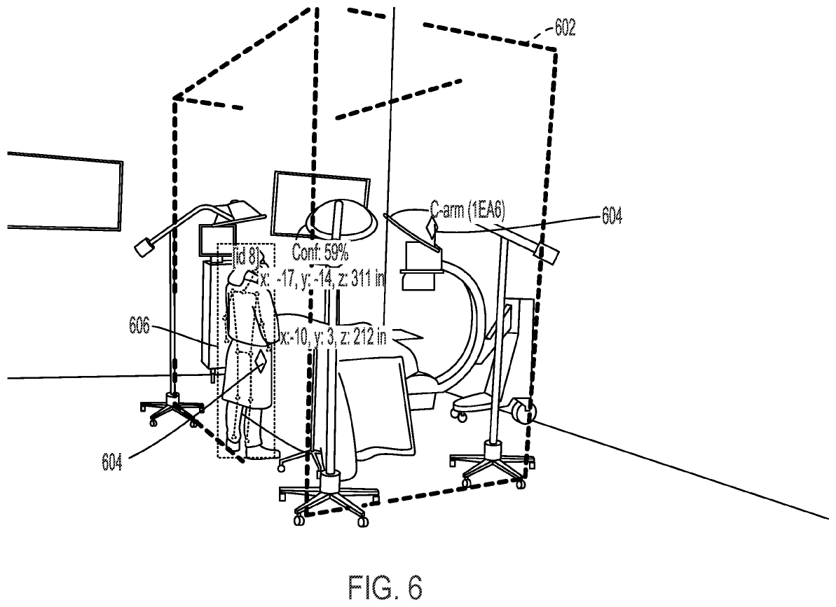

Example operations of the system include identifying virtual zones 602 (which may be two-dimensional or three-dimensional, as shown in FIG. 6), such as sterile zones around the surgical table and other critical zones in the room, augmented and overlaid onto the video data streams from cameras 108 using, e.g., cuboid algorithms to identify breach in zone from the AI-based location estimation of the human skeletal pose model in the room.

Example operations of the system include identifying hot spots through feeding and processing of thermal imaging data streams from thermal cameras 106.

Example operations of the system include detecting and tracking potential infection inception areas in the room by linking the location of the hot spots to one or more zones 602 from the underlying stereographic video data streams.

Example operations of the system include detecting and tracking breach of human movement into zones 602 by comparing the tracked human body motion in relation to the augmented, zones 602 to evaluate if the human skeletal pose model intersects with one of the zones 602.

Example operations of the system include measuring the weight of biohazard waste and general surgical procedure waste using load sensors 110.

Example operations of the system include detecting and tracking authorized door entries and exits by feeding and processing of the video data streams from cameras 108 to an AI via deep neural network and associate the human skeleton pose model and location within the video data stream for gathering accurate human body motion colliding with door zones defined in the system and, in a similar way, track unauthorized door entries and exits.

Example operations of the system include detecting and tracking door open and door close events based on a door signal from door sensors 112. Example operations of the system include tracking particle count using air particle sensors. Example operations of the system include tracking air refresh cycle using airflow sensors. Example operations of the system include transcribing speech to text by feeding audio signals from microphones 102 to an AI natural language processing model and audio voice transcription via deep neural networks and text enhancements using transformer deep neural networks to achieve textual transcriptions and perform speaker identification using an ensemble of classifier models.

Systematic collection of all potential adverse events during training and during actual surgical medical procedures provides a resource of information consistently collected enabling comparison and analytics that can be used at a later date to conduct epidemiologic assessments and then prevent adverse events from happening in the first place using real-time notifications and feedback.

In one or more embodiments, a system can include one or more of the sensors and/or devices discussed above, all connected through a network apparatus which itself can be connected to a cloud computer service. The system, through its multiple sensors, can have an aggregator subsystem combining the sensors inputs to feed them to at least one AI algorithm. The system can have the ability to define virtual, three-dimensional zones such as room door entries, room door exits, sterile surgical zones, and other three-dimensional zones of importance for analytics; all of which are then leveraged by the overall system's algorithms. The tracking of human body and hand motion is computed by feeding the three-dimensional stereographic data to an AI model. The resulting positions in three-dimensions in the room are then compared to virtual zones 602, such as a sterile surgical zone, for computing real-time breaching of the zones 602. In a similar way, a human body motion entering a door zone or exiting a door zone generates an event tracking these entries and exits. In combining virtual zones and sensor data fusion, the system is configured for tracking, detecting, and analyzing the potential adverse events happening within the entire surgical room through the tracking of at least the biometric movement of the users, users' hands, medical instruments, and medical devices being used to perform a medical procedure by at least one user over at least one body form within a surgical training space or a real surgical space. At least one central apparatus can be mounted in the medical training or surgical space. The apparatus can also be connected to one or more stereoscopic cameras 108, each with a thermal imaging camera 106, that are able to capture the user's biometric movement in the training or surgical space as well as thermal and environmental conditions of objects and people in the room, as one or more tasks are performed by at least one user. Each stereoscopic camera 108 and thermal imaging camera 106 can be connected to a dedicated edge microprocessor to capture the live video, live audio, live thermal data, and the spatial data generated by each camera. The system can use a dedicated pose detection engine on the edge microprocessor, which can be connected to a dedicated computer through an internal network connection, to compute and detect breach events. The computer can be operable through an administrative interface, and/or the cloud administrative interface, to invoke the recording and storage of the live video, audio, sensor telemetry, and spatial data captured from a starting capture point of the procedure, and concluding with an end capture point of the procedure over a variable time span determined by the user or automatically determined from the captured data.

In some examples, the sensors can be mounted in particular arrangement within a medical procedure room. For example, a stereoscopic camera 108, a thermal camera 106, a tracking sensor receiver 104, and a microphone 102 can be co-located in each corner of the room and directed towards the center of the room. In some examples, a module of sensors is positioned in each corner of the room. The module can be a housing that contains a stereoscopic camera 108, thermal camera 106, a tracing sensor receiver 104, and a microphone 102. In some examples, an additional stereoscopic camera 108 is located in the center of the room. For example, the additional camera can be mounted to the ceiling of the room and directed downwards at an operating table. In some examples, a module of sensors is mounted from the ceiling above the operating table and directed towards the operating table.

In some examples, coordinate systems for other sensors are calibrated to correspond with a coordinate system of the stereoscopic cameras 108. For example, a coordinate system for RTLS tracking sensors can be calibrated to correspond with the coordinate system of the stereoscopic cameras 108. An exemplary calibration process includes removing all or most equipment from a room, if needed. Placing an ArUco marker at the center of the room as a reference point for each camera. Calibrating a coordinate system based on video feeds from the stereoscopic cameras mounted in the corners of the room based on the ArUco marker at the center of the room. For instance, stereoscopic camera coordinate system can be calibrated based on the location and orientation of the ArUco marker. ArUco markers can also be placed on a plurality of RTLS sensors distributed around the room. The system can determine the position of each sensor relative to each RTLS receiver, and determine the location of each sensor within the video feeds of the cameras and within the video based coordinate system. The system can correlate the RTLS position data with the video position data based on the ArUco makers to calibrate the RTLS sensor system with the video based coordinate system of the cameras.

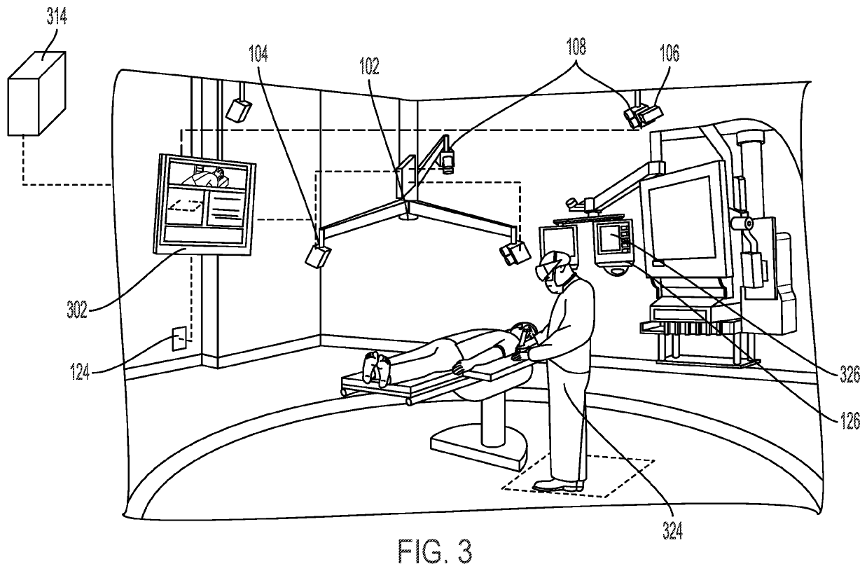

FIG. 3 shows an example operating room equipped with sensors and devices discussed above, such as microphones 102, UWB sensors 104, thermal cameras 106, stereographic cameras 108, and admin console 126. As shown, radio frequency identification (RFID) badges 324 can provide identification and tracking of personnel when entering a physical space, such as an operating or training room, and tracking of the person through the space based on tracking signals from UWB sensors 104 and identification from RFID reader 118 as the person crosses the entry threshold of the space. When an identification is made, the data can be captured and stored, including optionally to the cloud service 122, and presented to the admin console 126. The data can be displayed on a user interface 326 of the admin console 126 as well as on a heads-up display (HUD) 302 located locally in the room and/or an augmented reality (AR) headset. User interface 326 can present alerts to an administrative user for various events, including adverse events such as breaches, potential infections, instrument drops, and unauthorized entries. The alerts can be in real-time or near real-time and facilitate training and/or taking steps to prevent adverse events. HUD 302 can display various types of information, including for example, patient information, patient vital data, sensor data, case information, anesthesia time, time-out checklists, room monitoring, dosage readings, instrument count, and camera views of the surgical site. Admin console 126 can be directly connected to a server 314 through a secure network connection 124.

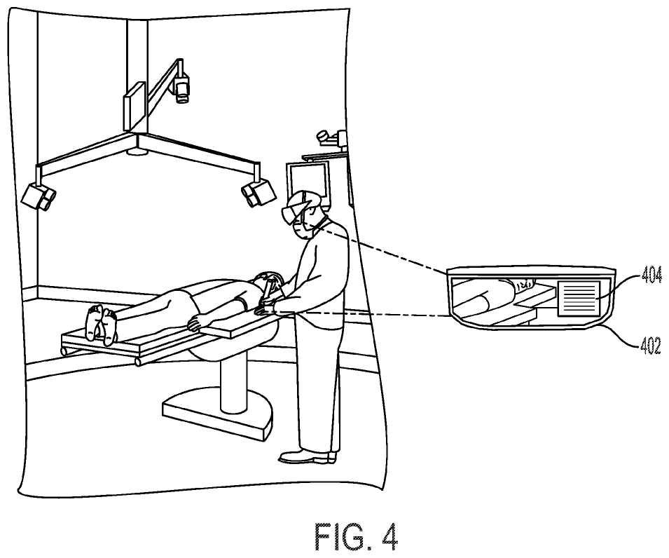

FIG. 4 shows a surgeon, student, or surgical staff member wearing an AR headset 402. When an identification is made, an AR display 404 can be shown in the AR headset 402. The AR display 404 can contain graphics related to various types of information, such as corrective action, patient information, environmental information, vitals, and time stamps.

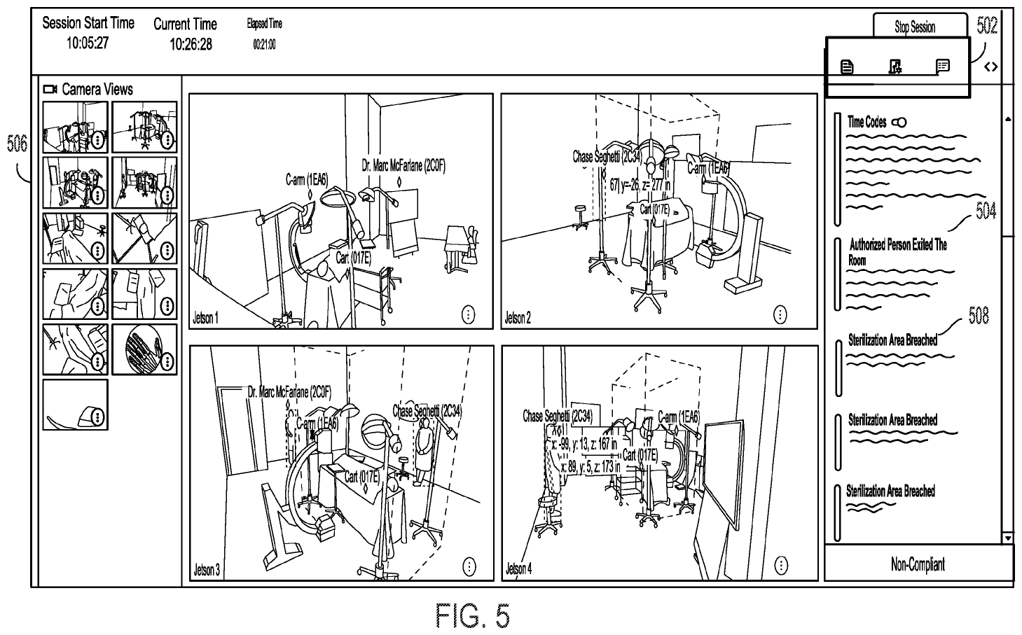

Referring to FIG. 5, one screen of the user interface 326 can allow the user to interact with all of the hardware in order to collect their desired data sets. Within user interface 326, a user can pin any of the imaging device displays 506. The user can also toggle between various data tabs 502, such as a sensor tab, a transcriptions tab, and an events tab. For example, during a live session, playback mode, or archive mode, a user can view alerts for an authorized person event 504 and/or a sterilization zone breach 508 on the events tab.

Referring to FIG. 6, another screen of the user interface 326 can display two-dimensional or three-dimensional virtual zones 602 overlaid on video data streams from cameras 108 and UWB markers 604 from UWB sensors 104, which spatially track movement of people and equipment. As also shown in FIG. 6, user interface 326 can display a human pose model 606, which identifies human skeletal points and displays a skeletal overlay on the video data stream from cameras 108. When an identified, unauthorized human pose model 606 enters into zone 602, the user interface can initiate a breach alert and change the color of zone 602 (e.g., from green or yellow, when unbreached, to red, when breached).

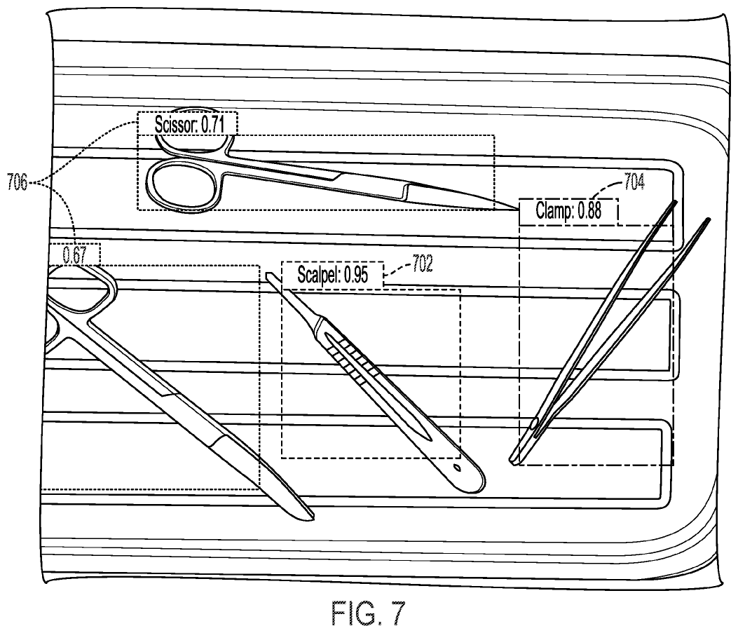

Referring to FIG. 7, yet another screen of user interface 326 can display identification and classification of various instruments, such as scalpel 702, clamp 704. and scissors 706, overlaid on video data streams from cameras 108. Examples of instruments that the system can identify include, without limitation, scissors (e.g., Mayo scissors, Metzenbaum scissors, Potts scissors, and iris scissors), pickups/forceps (e.g., tissue forceps, Adson forceps, Ferris-Smith forceps, Bonney forceps, DeBakey forceps, Kocher forceps, right angle forceps, and Russian forceps), clamps/hemostats (e.g., Crile hemostatic clamps, Kelly clamps, and Allis-Babcock clamps), retractors (e.g., rake retractors, Volkman sharp retractors, Richardson retractors, flat retractors, malleable retractors, pickle fork retractors, Army-Navy retractors, Deaver retractors, Z-retractors, Hohmann retractors, cobra retractors, scissor-esque retractors, Gelpi retractors, Weitlaner retractors, and Bookwalkter retractors), rongeurs (e.g., Adson rongeurs, and double action rongeurs), spreaders (e.g., Lamina spreaders), mallets (e.g., ortho heavy mallets), saws (e.g., bone saws), power equipment (e.g., power drills, reamers, and pin-drivers), pliers, bone hooks, metal rulers, chisels, osteotomes (e.g., Lambotte osteotomes), and laparoscopic instruments. User interface 326 can color code instruments by type, for example, scalpels 702 with pink boxes, clamps 704 with orange boxes, and scissors 706 with green boxes. The system can use video data streams from cameras 108 and audio signals from microphones 102 to detect when an instrument has been dropped, and user interface 326 can initiate a drop event to alert a user that a classified instrument has been dropped.

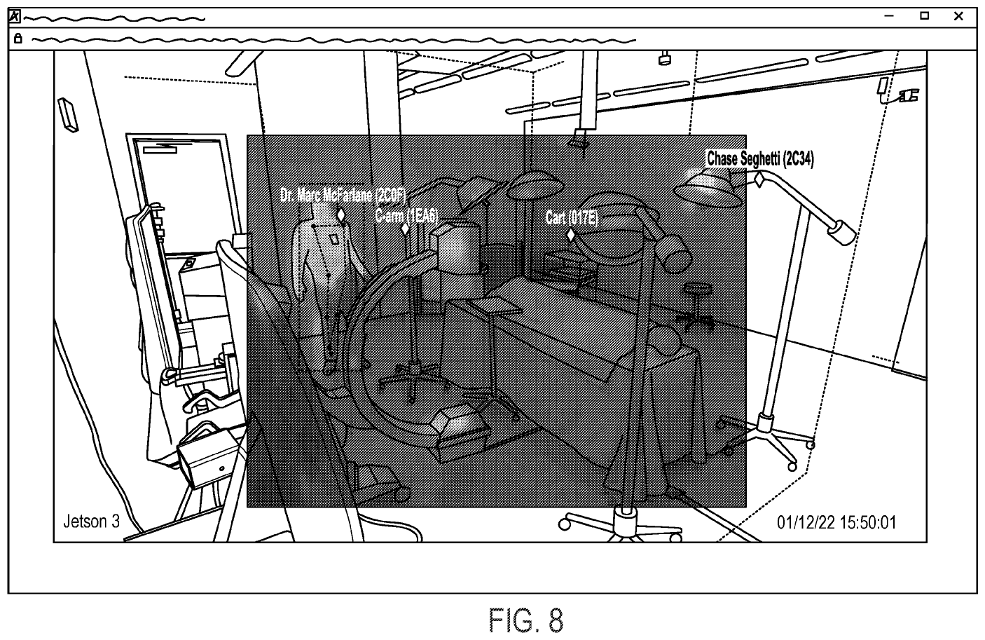

Referring to FIG. 8, a screen of user interface 326 can display thermal imaging from thermal cameras 106 and infrared technologies to show areas with increased heat that can relate or cause infection inception areas which can contribute to surgical site infections (SSI). Infrared thermography can be used for monitoring asset health in terms of heat and bacterial growth.

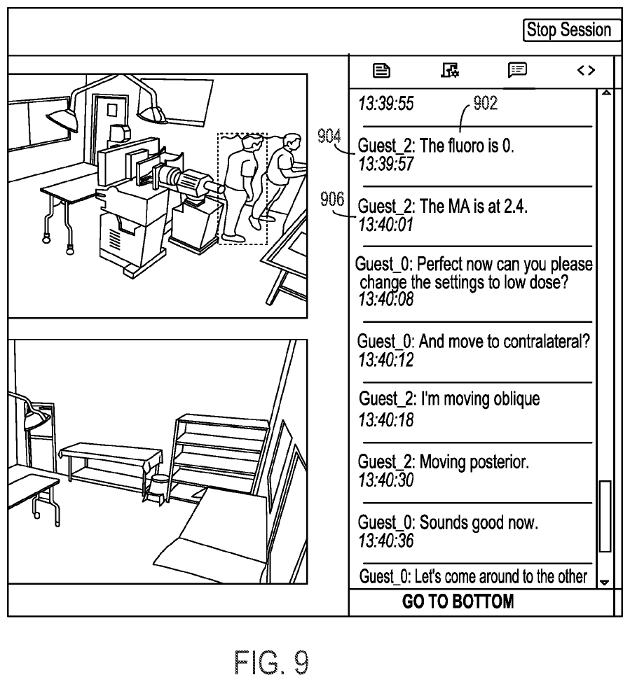

Referring to FIG. 9, a screen of user interface 326 can display speech transcriptions 902 based on audio signals from microphones 102. User interface 326 can also display a speaker identity 904 and a time stamp 906 for each spoken statement recorded by microphones 102.

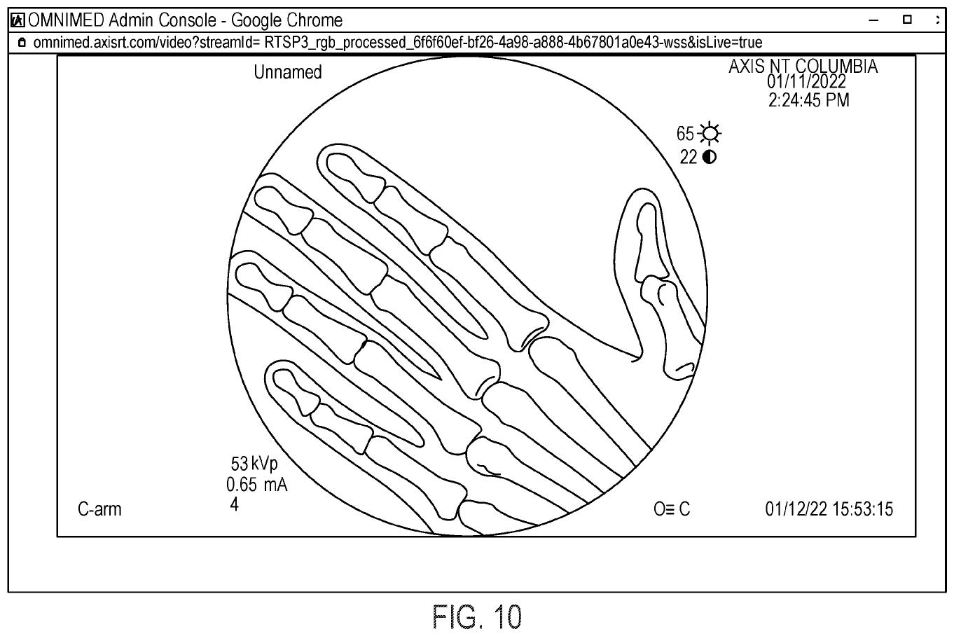

Referring to FIG. 10, a screen of user interface 326 can display views from medical imaging devices and equipment present in the room, such as from x-rays, c-arms, borescopes, and lap towers.

According to an embodiment of the present disclosure, user interface 326 can provide a playback mode to allow a user to rewind and pause the session while still in progress. User interface 326 can also provide an archive mode to allow a user to review a session that has completed.

According to another aspect of the present disclosure, the server 314 can be connected to cloud service 122 that analyzes the combined video, audio, sensor telemetry, and spatial data stream to generate augmented feedback in real time to the user or users in the room. Cloud service 122 can also include an analysis engine to analyze the data and develop data modeling utilizing a Naïve Bayes model, enabling efficient, contextual data analysis using classification algorithms. Cloud service 122 can also be operable to generate performance metrics for the plurality of people as the one or more tasks are performed based at least on the video from cameras 108, audio from microphones 102, sensor telemetry, and spatial data stream analysis of the biometric movement of the user, user hands, instrument movement, sensors telemetry, and spatial data.

According to another aspect of the present disclosure, the cloud service can also include a learning engine to develop augmented feedback created by the analysis engine to be delivered through the network 124 to augmented reality headset 402 worn by the user, which can display augmented feedback that is indicative of the quality of the real-time analyzed performance of the one or more users based at least on the position data as the tasks are performed. Once cause and effect of adverse patient outcomes are learned by one or more artificial intelligence engines, such as those discussed above, the feedback may include recommendations in near real-time for the user to take corrective actions to avoid adverse patient outcomes.

According to another aspect of the present disclosure, the cloud service 122 can utilize a computer administrative interface or the cloud administrative interface, enabling the administrative user to provision multiple users and capture and review video, audio, sensor telemetry, and spatial data captured and stored within the analysis engine and the learning engine to provide metrics indicative of the quality of performance of the one or more users based at least on the position data and related adverse event data collected by the system in real-time or in play-back mode.

According to another aspect of the present disclosure, the cloud service can utilize a content management engine that can be accessed through the admin interface on the computer, enabling the user to analyze and annotate stored video, audio, sensor telemetry, and spatial data captured by the system held within the learning engine, giving the user admin the ability to generate customized real time evaluation and augmented feedback to the user during the procedure.

Cloud service 122 can utilize a content management engine that can be accessed through admin console 126, enabling the user to analyze and annotate stored video, audio, sensor telemetry, and spatial data captured by the system held within the learning engine and giving the user the ability to review customized recorded evaluation and augmented feedback once the procedure is finalized in an archived mode.

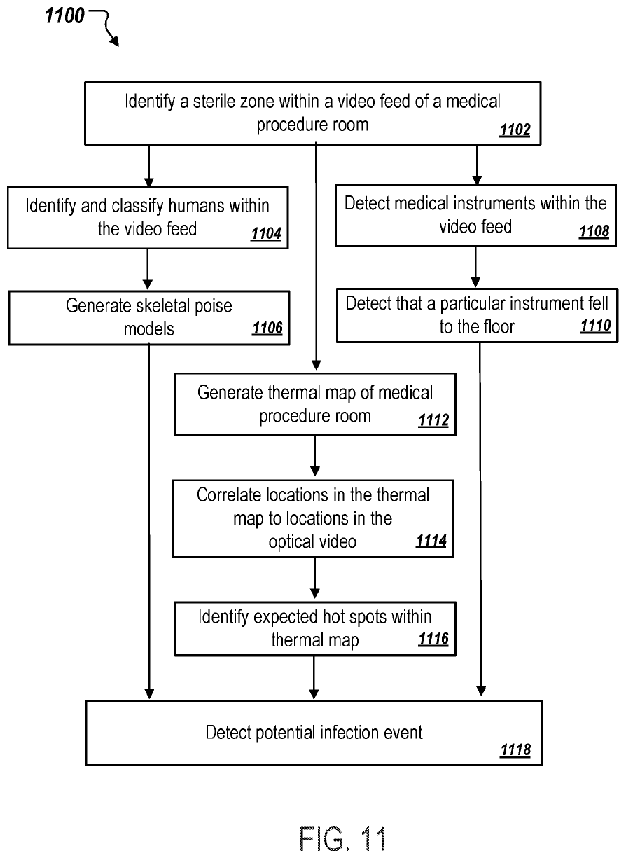

FIG. 11 depicts a flowchart of an example process 1100 for detecting events within a medical procedure room. Process 1100 can be executed by one or more computing systems including, e.g., the system and sensors described above. The system identifies a sterile zone within video feeds of a medical procedure room (e.g., operating room) (1102). For example, the system can obtain multiple video feeds from stereoscopic cameras mounted within the procedure room. In some examples the sterile zone is defined by a region of pixels within each video feed that represent a three-dimensional physical space surrounding a region of the room where invasive procedures are performed (e.g., around surgical table as depicted in FIG. 6). In some examples, the sterile zone can be a predefined region of within a particular room. In some examples, the system can identify, e.g., through object detection algorithms, the operating table within the room and generate a virtual sterile zone around the operating table based on standoff distances from the table. The standoff distances may be predefined distances.

The system identifies humans within the video feeds (1104). For example, the system can employ object detection and tracking algorithms as discussed above to identify humans within the video feeds. In some embodiments, the system can classify the humans as being permitted or not permitted to enter the sterile zone. For instance, a surgeon would be permitted to enter the sterile zone, but a nurse or hospital attendant may not be. Reducing the number of individuals entering the sterile zone reduces the potential for post operation infections and intrusion of unintended object and bacteria within the sterile zone. The system can classify individuals based on sensors worn by the individuals, e.g., RFID sensors, UWB sensors, other location tracking and identification sensors, or a combination thereof. For example, the system can classify individuals by determining an individual's identify from an RFID scan, e.g., a badge scan when entering the room. The system can determine the individual's permission status (e.g., permission to enter the sterile zone) based on their identify and/or a role associated with the individual, such as their position (e.g., surgeon, nurse, etc.). Once the individual's permission is determined, the system can tag the individual within the video feeds (e.g., the object representing the individual in the video feeds) with a metadata tag that indicates whether the individual is permitted to enter the sterile zone or not. In some implementations, the metadata tag is associated with a tracking sensor (e.g., UWB sensor) worn by the individual, and the system can correlate the UWB sensor identifier and the sensor's location with the individual within the video feeds. In some implementations, the tracking sensor itself can contain information indicating the individual's permission to enter the sterile zone, such that a metadata tag is unnecessary so long as the tracking sensor is readable.

The system generates a skeletal poise mode of individuals identified within the video feed (1106). For example, the system can generate a skeletal poise model of human's major limbs as discussed above and shown in FIG. 6. In some examples, the system can generate a detailed skeletal poise model of individuals within the sterile zone (e.g., the surgeon) that includes mappings to the individuals' fingers. The system uses the skeletal poise models to monitor for and detect breaches of the sterile zone. The system can detect a potential infection event (1118) by determining whether a limb of an unauthorized person breaches a boundary of the sterile zone. For instance, the skeletal poise models can be tagged with the metadata tags that indicate which individuals are permitted to enter the sterile zone. The skeletal poise models allow the system to track movement of each individual's limbs with respect to the sterile zone and determine if a limb of an unauthorized person breaches a boundary of the zone.

The system can detect medical instruments within the video feeds (1108). For instance, the system can employ object detection algorithms to locate individual medical instruments within the video feed. In some examples, the system can identify a type of each detected instrument. For example, the system can employ a YOLO machine learning model to analyze a region of pixels within one or more frames of the video feeds to identify instrument type. The system can track the location of the detected instruments. For example, the system can apply a metadata tag to detected instruments to uniquely identify each particular instrument and track its movements throughout the procedure. The system can employ and object tracking algorithm to monitor movement of each detected instrument through the multiple video feeds. In some implementations, the system also employs load or weight sensors on tables and/or carts upon which medical instruments may be set to aid in tracking the location of the medical instruments during a procedure. For example, when an instrument is tracked as being placed on a table or cart and, possibly, out of view of the cameras, the system can confirm that the instrument was placed on the table or cart based on a weight change corresponding to the weight of the instrument.

The system can detect that a particular instrument fell to the floor (1110). For example, by tracking the location of each instrument the system can detect when/if a particular instrument falls to the floor, therefor, becoming potentially contaminated. Such instrument should not enter the sterile zone. In some examples, the system can correlate video feed data with audio data (e.g., from microphones with the room) to detect a drop event. For instance, the system can employ a time correlation between video tracking and audio to confirm that a particular instrument has been dropped. The system can tag, or update a metadata tag of the instrument, to identify it as potentially contaminated. The system can detect a potential infection event (1118), by determining when an instrument tagged as potentially contaminated has crossed a boundary of the sterile zone within the video feeds.

The system can generate a thermal map of the medical procedure room (1112). For example, the system can obtain thermal video feds from thermal cameras located within the room, and from those video feeds, generate a thermal representation of the room and objects within the room. The system can correlate locations in the thermal map to locations in the optical video feeds (1114). For example, the system can correlate the location of hot spots within the thermal map to the optical video feeds from the stereoscopic cameras to identify the location of such hot spots relative to boundaries of the sterile zone. The system can correlate similar pixels within each video feed to determine the location of hot spots relative to appropriate boundaries of the sterile zone in the optical videos. In some implementations, pairs of stereoscopic and thermal cameras can be co-located within the room to aid in the correlation.

The system can “normalize” the thermal map, e.g., by identifying expected hot spots within the thermal map (1116). For instance, the system can identify expected hot spots within the sterile zone. A hot spot can be a region of pixels representing thermal radiation above a mean value for the map. In some examples, a hot spot can be a region of pixels representing thermal emission above a threshold value that represents a potential infection inception area. However, as depicted in FIG. 8, some hot spots are expected within the sterile zone, e.g., lights, machinery, and people. The system can identify such expected hot spots based on known signatures, e.g., temperature, shape, correlation with skeletal poise models. In some implementations, a machine learning algorithm, e.g., YOLO, may be used to identify the expected hot spots. The system can then detect a potential infection event (1118) when an unexpected hot spot is identified within the sterile zone. For example, due to unknown contamination an infection inception area may become apparent on an object within the sterile zone during surgery. Identifying, such an occurrence can enable professionals to quickly address the situation before the patient is infected.

Upon the detection of any potential infection event, the system can create an alert. For instance, as discussed above, the alert can be audible (e.g., an alarm), visual (e.g., presented on a display within the room), or both.

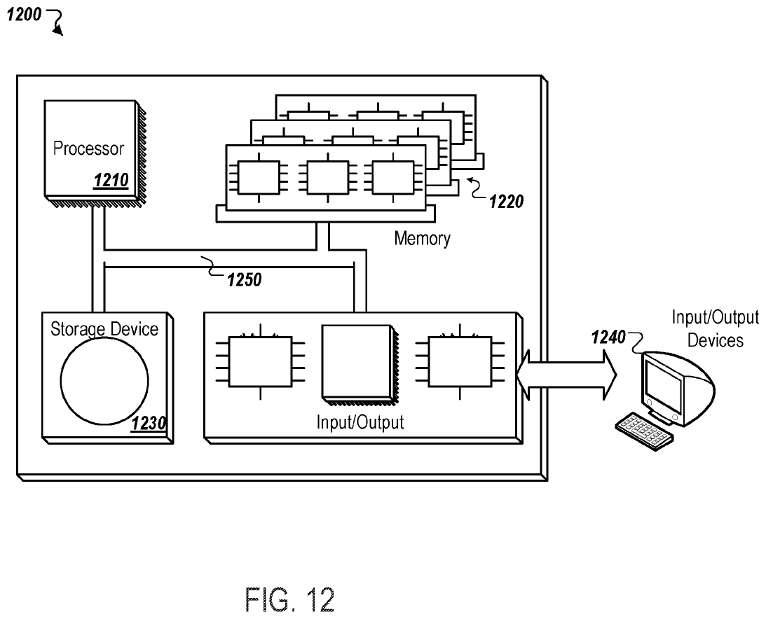

FIG. 12 is a schematic diagram of a computer system 1200. The system 1200 can be used to carry out the operations described in association with any of the computer-implemented methods described previously, according to some implementations. In some implementations, computing systems and devices and the functional operations described in this specification can be implemented in digital electronic circuitry, in tangibly-embodied computer software or firmware, in computer hardware, including the structures disclosed in this specification (e.g., system 1200) and their structural equivalents, or in combinations of one or more of them. The system 1200 is intended to include various forms of digital computers, such as laptops, desktops, workstations, personal digital assistants, servers, blade servers, mainframes, and other appropriate computers, including vehicles installed on base units or pod units of modular vehicles. The system 1200 can also include mobile devices, such as personal digital assistants, cellular telephones, smartphones, and other similar computing devices. Additionally, the system can include portable storage media, such as, Universal Serial Bus (USB) flash drives. For example, the USB flash drives may store operating systems and other applications. The USB flash drives can include input/output components, such as a wireless transducer or USB connector that may be inserted into a USB port of another computing device.

The system 1200 includes a processor 1210, a memory 1220, a storage device 1230, and an input/output device 1240. Each of the components 1210, 1220, 1230, and 1240 are interconnected using a system bus 1250. The processor 1210 is capable of processing instructions for execution within the system 1200. The processor may be designed using any of a number of architectures. For example, the processor 1210 may be a CISC (Complex Instruction Set Computers) processor, a RISC (Reduced Instruction Set Computer) processor, or a MISC (Minimal Instruction Set Computer) processor.

In one implementation, the processor 1210 is a single-threaded processor. In another implementation, the processor 1210 is a multi-threaded processor. The processor 1210 is capable of processing instructions stored in the memory 1220 or on the storage device 1230 to display graphical information for a user interface on the input/output device 1240.

The memory 1220 stores information within the system 1200. In one implementation, the memory 1220 is a computer-readable medium. In one implementation, the memory 1220 is a volatile memory unit. In another implementation, the memory 1220 is a non-volatile memory unit.

The storage device 1230 is capable of providing mass storage for the system 1200. In one implementation, the storage device 1230 is a computer-readable medium. In various different implementations, the storage device 1230 may be a floppy disk device, a hard disk device, an optical disk device, or a tape device.

The input/output device 1240 provides input/output operations for the system 1200. In one implementation, the input/output device 1240 includes a keyboard and/or pointing device. In another implementation, the input/output device 1240 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices. such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

While the present disclosure is described in the context of a psychological diagnostic system, it is understood that the techniques and processes described herein are applicable outside of this context. For example, the techniques and processes described herein may be applicable to other types of diagnostic machine learning systems including, but not limited to, medical diagnostic systems, computer software diagnostic (debugging) systems, computer hardware diagnostic systems, or quality assurance (e.g., in manufacturing) diagnostic systems.

1. A medical procedure tracking method executed by one or more processors, the method comprising:
identifying at least one sterile zone within video feeds obtained from at least a first stereoscopic camera and a second stereoscopic camera located within a medical procedure room, the sterile zone comprising a region of pixels that define a three-dimensional physical space surrounding a surgical table within the medical procedure room;
identifying, within the video feeds, humans in the medical procedure room and classifying each identified human as being permitted or not permitted to enter the sterile zone based on sensor data received from a tracking sensor worn by the human;
generating, for each identified human within the video feeds, a skeletal poise model and using the skeletal poise model in correlation with the tracking sensor data to track movement of the human's limbs with respect to the sterile zone;
determining that at least a portion of a skeletal poise model of at least one human classified as not being permitted to enter the sterile zone crossed a boundary of the sterile zone within at least one of the video feeds; and
in response, causing an alert to be presented on at least one display device located within the medical procedure room.

2. The method of claim 1, wherein the tracking sensor data is data from an ultra-wideband sensor worn by the human.

3. The method of claim 1, wherein classifying each identified human comprises:
determining an identity of the human based on data received from the tracking sensor;
determining, based on the identity, whether the human is permitted to enter the sterile zone; and
associating a metadata tag that indicates whether the human is permitted to enter the sterile zone with an identifier for the tracking sensor worn by the human.

4. The method of claim 1, wherein classifying each identified human comprises:
determining an identity of the human from radio frequency identification data received from a badge scan as the human enters a door of the medical procedure room;
determining, based on the identity, whether the human is permitted to enter the sterile zone; and
associating a metadata tag that indicates whether the human is permitted to enter the sterile zone to the skeletal poise model of the human.

5. The method of claim 1, wherein classifying each identified human comprises:
determining an identity of the human from radio frequency identification data received from a badge scan as the human enters a door of the medical procedure room;
determining, based on the identity, whether the human is permitted to enter the sterile zone; and
associating a metadata tag that indicates whether the human is permitted to enter the sterile zone with an identifier for the tracking sensor worn by the human.

6. The method of claim 5, wherein the tracking sensor is an ultra-wideband sensor.

7. The method of claim 1, further comprising:
detecting, within the video feeds, a plurality of medical instruments; and
for each medical instrument, identifying a type of the instrument.

8. The method of claim 7, wherein identifying the type of the instrument comprises using a YOLO machine learning model to analyze a region of pixels within one or more frames of the video feeds to identify the type of the instrument.

9. The method of claim 7, further comprising:
applying, to each detected medical instrument, a metadata tag to uniquely identify the respective instrument and the type of the instrument; and
monitoring the location of each detected instrument by tracking locations of each instrument within the video feeds using an object tracking algorithm in correlation with load sensor data received from one or more load sensors positioned on tables or carts used to temporarily store medical instruments during a medical procedure.

10. The method of claim 7, further comprising detecting that a particular medical instrument fell to a floor of the medical procedure room based on data within the video feeds and audio data received from one or more microphones positioned within the medical procedure room.

11. The method of claim 10, further comprising:
detecting a potential infection event responsive to tracking movement of the particular medical instrument within the video feeds and determining that the particular medical instrument crossed a boundary of the sterile zone within at least one of the video feeds; and
in response, causing an infection alert to be presented on at least one display device located within the medical procedure room.

12. The method of claim 10, further comprising:
generating, based on thermal video feeds from at least a first thermal camera and a second thermal camera located within the medical procedure room, a thermal map of the medical procedure room;
identifying a plurality of expected hot spots within the thermal map, the expected hot spots representing regions of elevated thermal emissions that are expected to be within the sterile zone;
detecting a new hot spot within the sterile zone; and
causing an infection alert to be presented on at least one display device located within the medical procedure room.

13. One or more non-transitory computer readable storage media storing instructions that, when executed by at least one processor, cause the at least one processor to perform the method of claim 1.

14. A medical procedure tracking system comprising:
a first stereoscopic camera and a second stereoscopic camera positioned within a medical procedure room;
a tracking sensor receiver positioned within the medical procedure room, the tracking sensor receiver configured to interface with tracking sensors to identify positions of the tracking sensors relative to a location of the tracking sensor within the medical procedure room;
one or more processors in electronic communication with the first stereoscopic camera and the second stereoscopic camera and the tracking sensor receiver; and
one or more tangible, non-transitory media operably connectable to the one or more processors and storing instructions that, when executed, perform to perform the method of claim 1.

15. A medical procedure room comprising:
a plurality of stereoscopic cameras arranged within the medical procedure room such that a surgical table within the medical procedure room is visible within a field of view of each stereoscopic camera from a different perspective;
a plurality of thermal cameras arranged within the medical procedure room such that the surgical table is visible within a field of view of each thermal camera from a different perspective;
at least one location tracking sensor receiver arranged within the medical procedure room to receive signals from location tracking sensors;
at least one microphone arranged within the medical procedure room to detect audio from a region surrounding the surgical table; and
at least one electronic display arranged within the medical procedure room such that images displayed on the display are visible from the surgical table,
wherein each of the a plurality of stereoscopic cameras, the plurality of thermal camera, the at least one location tracking sensor receiver, the at least one microphone, and the at least one electronic display are in electronic communication with one or more computers.

16. The medical procedure room of claim 15, further comprising:
a particular stereoscopic camera positioned within the medical procedure room to capture video of a sink located in the medical procedure room within a field of view of the particular camera; and
a second microphone located proximate to the sink.

17. A medical procedure room comprising:
a set of co-located sensors mounted in each corner of the medical procedure room and directed towards a center of the room, each set of co-located sensors comprising a stereoscopic camera, a thermal camera, a location tracking sensor, and a microphone; and
at least one electronic display arranged within the medical procedure room such that images displayed on the display are visible from a surgical table in the medical procedure room,
wherein each set of co-located sensors and the at least one electronic display are in electronic communication with one or more computers.
